(19) United States
(12) Patent Application Publication
(10) Pub. No.: US 2014/0323755 A1
(43) Pub. Date: Oct. 30, 2014

(54) Method for Producing Biodiesel Using Microorganisms Without Drying Process

(71) Applicant: Korea Research Institute of Bioscience and Biotechnology, Daejeon (KR)

(72) Inventors: Hee-Mock Oh, Daejeon (KR); Hyun Joon La, Daejon (KR); Jae Yon Lee, Daejon (KR); Hee-Sik Kim, Daejon (KR); Chi-Yong Ahn, Daejon (KR)

(21) Appl. No.: 14/358,186

(22) PCT Filed: May 3, 2013
(86) PCT No.: PCT/KR2013/003871

§ 371 Date: May 14, 2014

(30) Foreign Application Priority Data
May 4, 2012 (KR) 10-2012-0047487
May 3, 2013 (KR) 10-2013-0049934

(51) Int. Cl. C10L 1/02; C12P 7/64

(52) U.S. Cl. CPC C10L 1/02; C12P 7/649
USPC 560/129; 435/134

(57) ABSTRACT

There is provided a method of producing biodiesel without drying and lipid component extraction steps in an alcohol-rich condition. Also, there is provided a method of effectively producing biodiesel without a catalyst under optimal conditions for transesterification. Production cost and time are reduced by reducing the number of processes, and biodiesel yield is increased.

### CROSS-REFERENCE TO RELATED APPLICATION

This application claims priority to and the benefit of Korean Patent Application No. 10-2012-0047487, filed on May 4, 2012, the disclosure of which is incorporated herein by reference in its entirety.

### BACKGROUND

1. Field of the Invention

The present invention relates to a method of producing biodiesel, in which extraction and transesterification reactions of lipid components in wet microorganisms including microalgae and oleaginous microorganisms are performed at the same time, without drying and lipid extraction steps at ambient conditions.

2. Discussion of Related Art

Biodiesel is a fatty acid methyl ester (FAME) that is a non-polluting fuel prepared from a vegetable oil, microalgae, and oleaginous microorganisms etc. as feed stocks, and has a purity of 95% or higher. Biodiesel may be used as an additive for a diesel vehicle or as fuel of a general vehicle due to its similar physical properties to diesel.

Biodiesel has an environment improvement effect of reducing air pollution and greenhouse gases caused by the use of existing fossil energy. Furthermore, biodiesel is produced from a reusable biomass, and therefore avoids potential problems such as depletion of energy resources. In the case of biodiesel, net emission of carbon dioxide, which is causing global warming, is very small because carbon dioxide is removed during the production of a biomass. Also, biodiesel has a high perfect combustion ratio due to high oxygen content (at least 10% oxygen), is capable of reducing carcinogenic particulate matters, and produces less environmental pollution in case of leakage due to its low toxicity and high biodegradability.

Though there are differences depending on the species, microalgae can be anatomically divided into cell walls that contain high amounts of fiber and cytoplasm that contains a variety of materials. However, Lipids of some species are very suitable for preparing bio-fuels because they are significantly similar to vegetable oils. A biomass of microalgae contains lipids at 80% or less, carbohydrates at 20 to 40%, proteins at 30 to 70%, and some species also have lipid contents up to 80% of a dry weight (see “Biodiesel Production Technology Using Microalgae Marine Biomass,” KSBB journal 2010, 25: 109-115).

Microalgae fibers are mainly cellulose and have a relatively uniform diameter compared to plant-based cellulose fibers. Therefore, microalgae fibers may avoid disadvantage caused by change of physical properties of composite materials due to non-uniform size of cellulose in one fiber, which is a known problem of plant cellulose. A general method of producing biodiesel, bio-ethanol, bio-butanol, organic acids, and the like from microalgae on a laboratory scale is as follows. After first culturing microalgae, in order to purify biodiesel, bio-ethanol, and organic acids, most moisture in the microalgae is removed through centrifugation, filtering, and drying steps, after which lipids are extracted using a solvent with high selectivity to lipids, and then the extracted lipids are converted into biodiesel. Alternatively, the microalgae are fermented using suitable enzymes and mircoorganisms to produce bio-ethanol or an organic acid (e.g. lactic acid).

In a conventional biodiesel production process, cultured microalgae are harvested to obtain a microalgae powder through a drying step, lipids are extracted from the dried powder using a solvent, and the extracted lipids are subjected to alkali- or acid-catalyst assisted transesterification to produce a FAME (fatty acid methyl ester). Since the existing biodiesel conversion process involves drying and lipid extraction steps after harvesting microalgae, the process is complicated and costly.

In addition to microalgae, there is a method of producing biodiesel on a commercial scale from vegetable oils or animal oils. The method, which is widely known, includes adding methoxide to heated lipid components and allowing them to react for about 20 to 60 minutes to obtain a FAME. The method also requires at least two steps of reactions to obtain a FAME, because lipid components have to be separated from a plant or an animal.

The present inventors have developed a method of eliminating drying and lipid extraction steps and still increasing production of biodiesel at room temperature and normal pressure. Further, the present inventors have developed a method of producing biodiesel whereby transesterification can be effectively performed without a catalyst, thereby simplifying the biodiesel production process and considerably reducing costs.

### SUMMARY OF THE INVENTION

The present invention is directed to providing a method of producing biodiesel.

The present invention is also directed to providing biodiesel produced without a catalyst.

One aspect of the present invention provides a method of producing biodiesel, including:

1) culturing microorganisms and centrifuging the culture to obtain a pellet;

2) adding the pellet of step 1) to an alkyl alcohol and performing a transesterification reaction; and

3) extracting a fatty acid methyl ester (FAME) from the reaction product of step 2).

The microorganisms of step 1) may be at least one selected from the group consisting microalgae, yeast, bacteria, and fungi.

The pellet of step 1) may have a moisture content of 80 wt % to 98 wt %.

The alkyl alcohol of step 2) may be added in an amount of 10 to 1,000 mL per 1 g of dry weight of the pellet.

The method may further include mixing the pellet with the alkyl alcohol, and dispersing the pellet in the alkyl alcohol after adding the pellet of step 2) to the alkyl alcohol.

The alkyl alcohol may be methanol or ethanol.

The purity of alkyl alcohol can be down to 70% (major impurity is water).

The method may further include adding a catalyst to the pellet during the transesterification reaction of step 2).

The catalyst may be a solid catalyst.

The solid catalyst may be an alkali catalyst, a metal oxide, an alloy catalyst or mixture of aforementioned materials.

The alkali catalyst may be at least one selected from the group consisting of sodium hydroxide, potassium hydroxide, calcium hydroxide, magnesium hydroxide, aluminum hydroxide, barium hydroxide, iron hydroxide, lithium hydroxide, zinc hydroxide, nickel hydroxide, tin hydroxide, cobalt hydroxide, chromium hydroxide, ammonium hydroxide, zirconium hydroxide, titanium hydroxide, tantalum hydroxide, hafnium hydroxide, niobium hydroxide, and vanadium hydroxide, but is not limited thereto.

The metal oxide may be at least one selected from the group consisting of calcium oxide, magnesium oxide, strontium oxide, barium oxide, iron (II, III) oxide, aluminum oxide, copper oxide, sodium oxide, silicon dioxide, titanium oxide, tin oxide, zinc oxide, zirconium oxide, cerium oxide, lithium oxide, silver oxide, and antimony oxide, but is not limited thereto.

The alloy catalyst may be a catalyst used in a methanol-based fuel cell, but is not limited thereto.

The catalyst may be added in an amount of 0.01 to 10 g per 1 g of dry weight of the pellet, but the amount is not limited thereto.

The transesterification reaction of step 2) may be performed at 3 to 85° C. and 50 to 350 rpm, and pressure of a closed reaction system may be 0.5 to 1.5 bars, but the temperature and pressure are not limited thereto.

The method may further include recovering a magnetic metal oxide using an electromagnet, subjecting the magnetic metal oxide to heat treatment, and continually reusing the recycled metal catalyst after the transesterification reaction of step 2).

Another aspect of the present invention provides use of biodiesel produced by the biodiesel production method according to the present invention.

Phytol can be effectively produced by present invention.

### BRIEF DESCRIPTION OF THE DRAWINGS

The above and other objects, features, and advantages of the present invention will become more apparent to those of ordinary skill in the art by describing in detail exemplary embodiments thereof with reference to the attached drawings, in which:

FIG. 1 is a photograph showing a handmade reactor;

FIG. 2A is a graph showing an amount (mg/g) of a fatty acid methyl ester (FAME) produced according to a biomass state, amount of catalyst, and catalyst state;

FIG. 2B is a graph showing an amount (% of DCW) of a FAME produced according to a biomass state, amount of catalyst, and catalyst state;

FIG. 3A is a graph showing an amount (mg/g) of a FAME produced according to types of catalysts;

FIG. 3B is a graph showing an amount (% of DCW) of a FAME produced according to types of catalysts;

FIG. 4A is a graph showing an amount of a FAME produced according to an amount of catalyst and amount of biomass, by analyzing optimal conditions affecting a transesterification reaction through response surface methodology (RSM);

FIG. 4B is a graph showing an amount of a FAME produced according to an amount of catalyst and temperature, by analyzing optimal conditions affecting a transesterification reaction through RSM;

FIG. 4C is a graph showing an amount of a FAME produced according to a biomass-catalyst ratio and temperature, by analyzing optimal conditions affecting a transesterification reaction through RSM;

FIG. 4D is a graph showing an amount of a FAME produced according to an amount of a biomass and temperature, by analyzing optimal conditions affecting a transesterification reaction through RSM;

FIG. 4E is a graph showing a saponification coefficient according to a biomass-catalyst ratio and an amount of a FAME produced, by analyzing optimal conditions affecting a transesterification reaction through RSM;

FIG. 5 is a graph showing an amount of a FAME produced and biodiesel components according to an amount of catalyst; and

FIG. 6 is a graph showing a FAME produced by using a yeast biomass under optimal reaction conditions deduced through RSM.

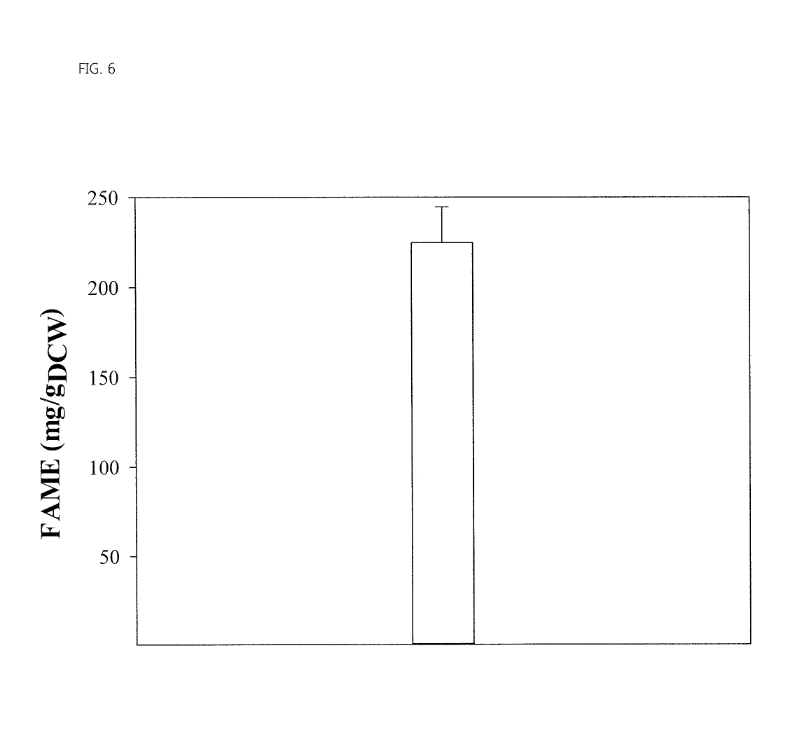

### DETAILED DESCRIPTION OF EXEMPLARY EMBODIMENTS

The term “biomass” used in the present specification refers to an organism or organisms used as an energy source.

The term “fatty acid methyl ester (FAME)” used in the present specification refers to a major component of biodiesel and may be used interchangeably with biodiesel.

The term “wet biomass” used in the present specification refers to a pellet generated only by centrifugation without a drying step after culturing microorganisms.

The term “dry biomass” used in the present specification refers to a pellet from which moisture is removed through a drying step after culturing microorganisms.

The term “transesterification” used in the present specification refers to a reaction which converts lipids of microorganisms into a FAME.

A method for the production of biodiesel is provided, including:

1) culturing microorganisms and centrifuging the culture to obtain a pellet;

2) adding an alkyl alcohol to the pellet of step 1) and performing a transesterification reaction; and

3) extracting a FAME from the reaction product of step 2).

The microorganisms may be photosynthetic microorganisms or oleaginous microorganisms. Also, algae, yeast, bacteria, and fungi having different lipid components or FAME profiles may be used as feed stocks for FAME production, and thus lipid components in various living bodies may be effectively converted into biodiesel.

The algae are preferably selected from microalgae, the yeast is preferably selected from Yarrowia, and the fungi are preferably selected from Aureobasidium pullulans, but these are not limitations.

The pellet of step 1) preferably has a moisture content of 80 wt % to 98 wt %, but is not limited thereto.

The centrifugation is preferably performed at 3000 to 5000 rpm for 1 to 10 minutes, but is not limited thereto.

The alkyl alcohol of step 2) is preferably added in an amount of 10 to 10,000 mL per 1 g of dry weight of a pellet (wet biomass), but the amount is not limited thereto. Dry weight of a wet biomass is a value of the wet biomass in terms of dry cell weight (DCW).

The alkyl alcohol is preferably methanol or ethanol, and more preferably methanol, but is not limited thereto.

The alkyl alcohol is reacted using a solid catalyst to form a strong base such as methoxide or ethoxide, thus inducing transesterification, a form of nucleophilic substitution. Therefore, when a solid catalyst able to excellently remove protons in an alcohol is reacted in-situ with microorganisms in an alcohol-rich condition, lipid components may be extracted at a high temperature and subjected to transesterification by a strong base formed by reaction with the solid catalyst.

The pellet of step 2) is preferably added to an alkyl alcohol, followed by mixing and dispersion, but this is not a limitation.

The method may further include adding a catalyst to the pellet during the transesterification reaction of step 2).

The catalyst is preferably a solid catalyst, but is not limited thereto.

The higher FAME yield may come from the transesterification of other lipids compounds such as phospholipids, galactolipids than neutral lipids. It has been reported that some transesterification methods performed in excess methanol often show higher FAME yield than conventional transesterification processes because of the transesterification of aforementioned cellular lipids.

When an amount of the alkyl alcohol is sufficiently greater than that of the biomass or lipid, saponification, which competes with transesterification, is suppressed and may be minimized due to an alkyl alcohol-rich condition.

The catalyst is preferably an alkali catalyst, a metal oxide, or an alloy catalyst, but is not limited thereto.

The alkali catalyst is preferably at least one selected from the group consisting of sodium hydroxide, potassium hydroxide, calcium hydroxide, magnesium hydroxide, aluminum hydroxide, barium hydroxide, iron hydroxide, lithium hydroxide, zinc hydroxide, nickel hydroxide, tin hydroxide, cobalt hydroxide, chromium hydroxide, ammonium hydroxide, zirconium hydroxide, titanium hydroxide, tantalum hydroxide, hafnium hydroxide, niobium hydroxide, and vanadium hydroxide, but is not limited thereto.

The metal oxide is preferably at least one selected from the group consisting of calcium oxide, magnesium oxide, strontium oxide, barium oxide, iron (II, III) oxide, aluminum oxide, copper oxide, sodium oxide, silicon dioxide, titanium oxide, tin oxide, zinc oxide, zirconium oxide, cerium oxide, lithium oxide, silver oxide, and antimony oxide.

The alloy catalyst is preferably a catalyst used in a methanol-based fuel cell, but is not limited thereto.

The catalyst is preferably added in an amount of 0.01 to 10 g per 1 g of dry weight of a pellet, but the amount is not limited thereto.

The transesterification reaction of step 2) is preferably performed at 4 to 60° C. and 50 to 350 rpm, but the temperature and pressure are not limited thereto.

When a magnetic metal oxide is added as a catalyst, the method of producing biodiesel according to the present invention may further include recovering a metal catalyst using an electromagnet, subjecting the catalyst to heat treatment, and continually reusing the recycled metal catalyst after the transesterification reaction. The magnetic metal oxide may be an iron oxide (Fe₂O₃), an Nb—Ti alloy, or the like, but is not limited thereto.

The extracting of the FAME of step 3) may include extracting the FAME by various extraction methods which are known in the related art, preferably using an organic solvent, separating a FAME-solvent, and filtering the FAME using an organic solvent filter, but these are not limitations.

Also, the present invention provides use of biodiesel produced by the method.

Hereinafter, the present invention will be described in more detail with reference to Examples. These Examples should not be misconstrued as limiting the scope of the present invention. Examples are provided to fully describing the present invention to those of ordinary skill in the art.

### EXAMPLES

### Example 1

### Method of Producing Biodiesel without Drying and Lipid Extraction Steps

### <1-1> Microalgae Culture

In order to prepare a FAME used as biodiesel, the microalgae Chlorella vulgaris AG10032 (provided by Biological Resource Center (BRC), Korea) were cultured for 14 days in a BG11 medium (see Rippka, R., DeReuelles, J., Waterbury, J. B., Herdman, M. & Stanier, R. Y. (1979). Generic assignments, strain histories and properties of pure cultures of cyanobacteria. J Gen Microbiol 111, 1-61) in a 7 L jar fermentor supplied with air at a rate of 0.1 v/v/m and irradiated by light at 120 μmol m⁻² s⁻¹. A dry cell weight of the cultured microalgae was measured. 50 mL of the cultured microalgae was centrifuged at 4000 rpm for 5 minutes at 25° C. using a 50 mL conical tube, and then the supernatant was removed to obtain a pellet (a wet biomass with a dry weight of about 0.1 g).

### <1-2> Transesterification Reaction

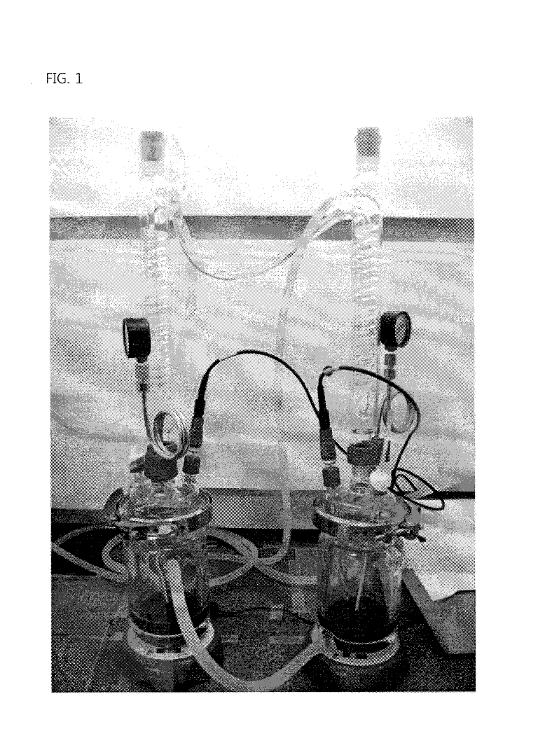

0.1 g of the pellet (i.e., wet biomass, DCW basis) obtained in Example <1-1> was added to 500 mL of a handmade double jacket reactor (FIG. 1) without drying and lipid extraction steps, and then 100 mL of methanol and a catalyst (NaOH, manufactured by Sigma Corporation) were added thereto under the conditions described in Table 1. Subsequently, the mixture was reacted for 60 minutes while stirring at 300 rpm at room temperature, 25° C. A condenser was provided on a cover of the handmade double jacket reactor to circulate water, and thus loss of a reaction liquid caused by internal and external heat was minimized.

Further, as a control of the wet biomass, the pellet obtained in Example <1-1> was freeze-dried to completely remove moisture (i.e., drying step), to obtain a biomass in a dry state, 0.1 g of the dry biomass was added to 500 mL of a double jacket reactor, and then 100 mL of methanol and a catalyst were added thereto under the conditions described in Table 1. Subsequently, the mixture was reacted for 60 minutes while stirring at 300 rpm at room temperature, 25° C.

TABLE 1

| | | Amount | | | |
|---|---|---|---|---|---|
| | | of | Reaction | Reaction | Stirring |
| Biomass | Type of | catalyst | temperature | time | rate |
| state | Catalyst | (g) | (° C.) | (min) | (rpm) |
| Dry | Solid pellet | 0.1 | 25 | 60 | 300 |
| | type NaOH | | | | |
| Wet | — | — | 25 | 60 | 300 |
| Wet | Solid, bead | 0.01 | 25 | 60 | 300 |
| | type NaOH | | | | |
| Wet | Solid, bead | 0.02 | 25 | 60 | 300 |
| | type NaOH | | | | |
| Wet | Solid, bead | 0.05 | 25 | 60 | 300 |
| | type NaOH | | | | |
| Wet | Solid, pellet | 0.1 | 25 | 60 | 300 |
| | type NaOH | | | | |
| Wet | Solid, pellet | 0.2 | 25 | 60 | 300 |
| | type NaOH | | | | |
| Wet | Solid, pellet | 0.5 | 25 | 60 | 300 |
| | type NaOH | | | | |
| Wet | Solid, pellet | 1 | 25 | 60 | 300 |
| | type NaOH | | | | |
| Wet | 20N NaOH | 0.1 | 25 | 60 | 300 |
| | solution | | | | |
| Wet | 22N NaOH | 0.2 | 25 | 60 | 300 |
| | solution | | | | |
| Wet | 24N NaOH | 0.5 | 25 | 60 | 300 |
| | solution | | | | |

*wet: Moisture content of 82 to 85 wt %

### <1-3> Analysis of FAME

After the transesterification reaction of Example <1-2>, 25 mL of the reaction liquid was transferred into a conical tube, 10 mL of an extraction solvent in which hexane and tert-butyl methyl ether were mixed at a volume ratio of 1:1 was added thereto, and then a FAME was extracted from the reaction liquid. 5 mL of a 4 N sodium hydroxide solution was further added to the extracted FAME to induce separation of a FAME-solvent layer. 1 mL was taken from the separated supernatant, the FAME-solvent layer was filtered using a polytetrafluoroethylene (PTFE) organic solvent filter, and then transferred to a GC vial, and then 50 μL of C17 internal standard material (manufactured by Fluka Chemical Corp.) was added thereto to prepare an assay sample for FAME content analysis. The FAME (or biodiesel) was analyzed by gas chromatography (Shimadzu GC-2010, Japan) and detected using an Rt-wax column (maximum temperature: 250° C.) and a flame ionization detector (FID, maximum temperature: 300° C.). 1 μL of each sample was injected to the GC. The detection time was set to 30 minutes. FAME mix 18918 (c8-c24, Supelco, Inc) was used as a standard material for the peak identification in GC analysis. Each peak of a sample was identified by comparing with the retention time of peaks obtained from the standard material, and then was quantified.

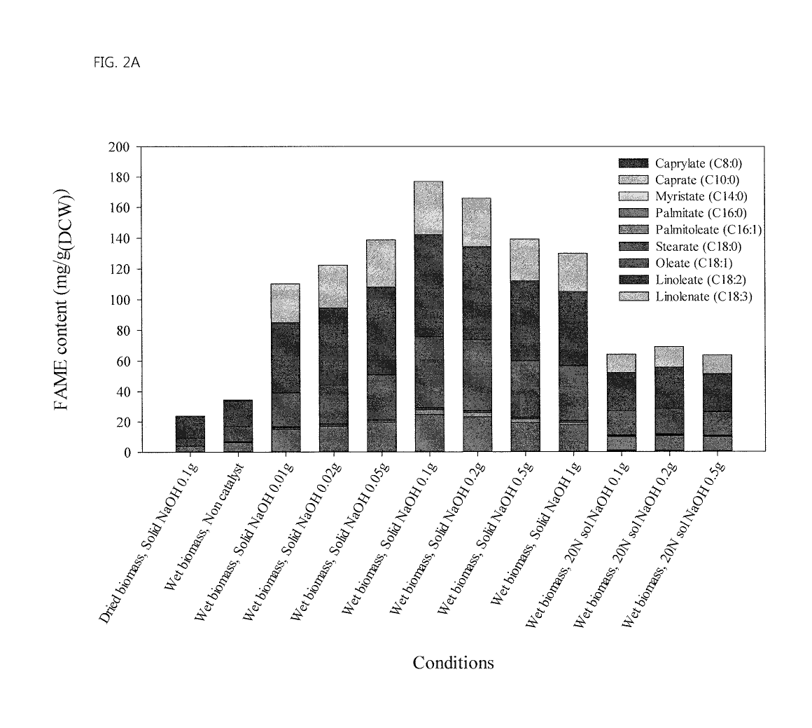

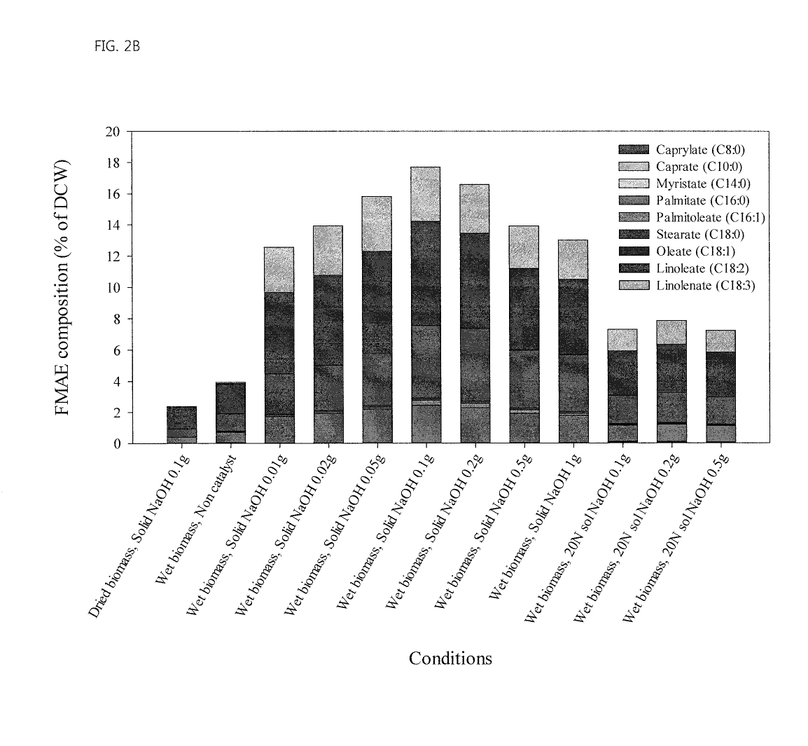

As a result, the amount of biodiesel according to reaction conditions of Table 1 (biomass state (dry, wet), types of catalysts (solid, solution), and reaction temperature) showed that the FAME yield obtained using a dry biomass was 30 mg/g (DCW) or less, which was the lowest among all reaction conditions and about ⅙ of the highest amount of biodiesel obtained using a wet biomass (detection of 180 mg/g in a condition of 0.1 g of solid pellet type NaOH) (FIG. 2). This is believed to be because dried biomass particles aggregated together such that methanol was prohibited from permeating into cells, and thus extraction efficiency of lipid components in the cells was reduced significantly, the reaction rate of lipid components with a reaction catalyst, sodium methoxide produced by the bond of methanol and sodium hydroxide was reduced, and thereby the amount of biodiesel produced was reduced.

Further, when sodium hydroxide in a liquid phase was used, the amount of biodiesel produced averaged 79 mg/g (DCW). When a catalyst in a solid phase was used, the amount of biodiesel produced averaged 146 mg/g (DCW). Hence, the amount of the biodiesel obtained using a catalyst in a liquid phase was about half the amount of biodiesel obtained using a catalyst in a solid phase, and therefore the solid catalyst had higher efficiency. When 0.1 g of the catalyst in a solid phase was used, the amount of biodiesel produced was the highest (FIG. 2).

Also, when the amount of catalyst was more than a predetermined level (0.1 g), it was found that the amount of the FAME produced was reduced.

### <1-4> Comparison of Amounts of Biodiesels Produced According to Types of Solid Catalysts

The amounts of the biodiesels produced according to types of solid catalysts were compared.

Specifically, 0.1 g of the pellet (i.e., wet biomass) obtained in Example <1-1> was added to 500 mL of a handmade double jacket reactor, and then 100 mL of methanol and a metal oxide (CaO, MgO, SrO, and Fe₂O₃; manufactured by Sigma Corporation) were added thereto according to the type and molar ratio of NaOH (0.5 g of biomass per 0.2 g of NaOH) under the conditions described in Table 2. Subsequently, the mixture was subjected to transesterification for 1 hour at room temperature, and then the biodiesel was extracted in a similar manner to Example <1-3> to measure the amount of biodiesel produced.

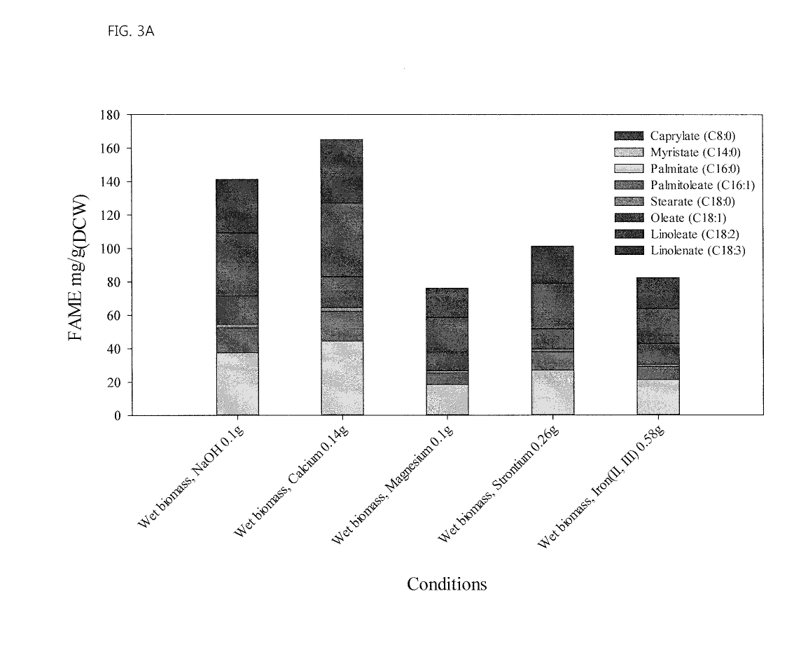

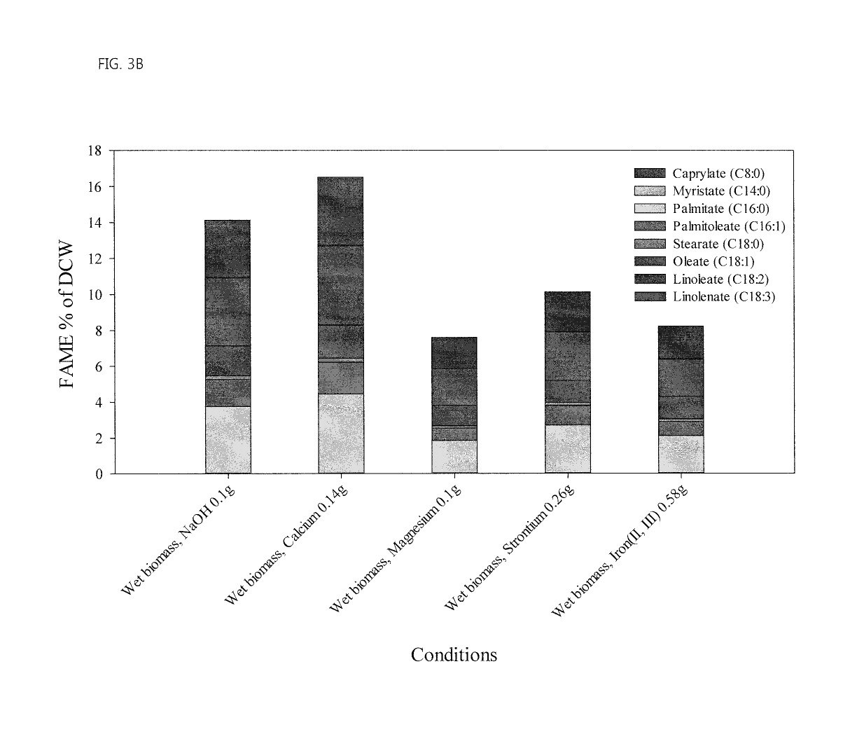

As a result, when sodium hydroxide or calcium oxide was used as a solid catalyst, the amounts of biodiesel were the highest (i.e., 140 mg/g (DCW) or more). When magnesium oxide, strontium oxide, or iron oxide was used, the amounts were 100 mg/g (DCW) or less, and efficiency was low (FIG. 3).

TABLE 2

| | | Amount | Reaction | Reaction | Stirring |
|---|---|---|---|---|---|
| Sample | Type of | of catalyst | temperature | time | rate |
| state | catalyst | (g) | (° C.) | (min) | (rpm) |
| Wet | Solid, pellet | 0.1 | 25 | 60 | 300 |
| | type NaOH | | | | |
| Wet | Solid, pellet | 0.14 | 25 | 60 | 300 |
| | type CaO | | | | |
| Wet | Solid, pellet | 0.1 | 25 | 60 | 300 |
| | type MgO | | | | |
| Wet | Solid, pellet | 0.26 | 25 | 60 | 300 |
| | type SrO | | | | |
| Wet | Solid, pellet | 0.58 | 25 | 60 | 300 |
| | type Fe₂O₃ | | | | |

For reference, samples used in Example <1-3> and Example <1-4> were cultured for different lengths of time. Thus, while the lipid content thereof may differ according to the different states of the cultured biomass and thus the amounts of biodiesel converted may also differ, there is no difference in conversion efficiency.

### Example 2

### Response Surface Methodology (RSM) Analysis for Optimization of Transesterification

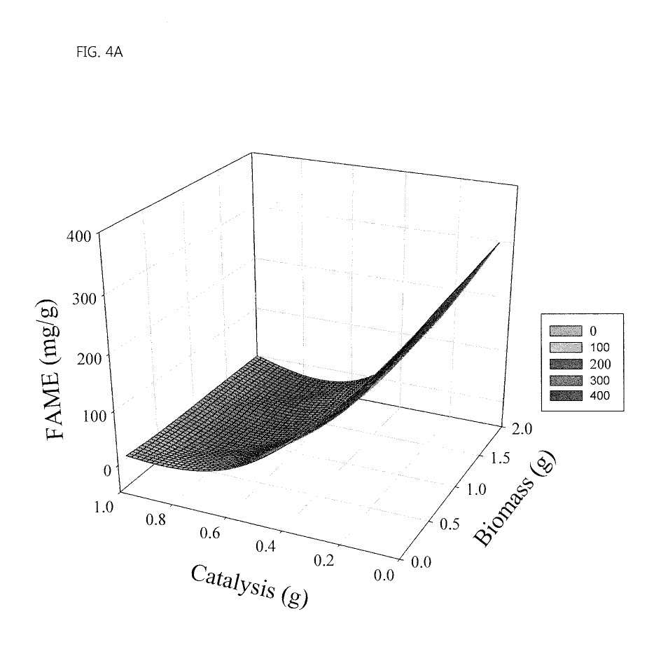

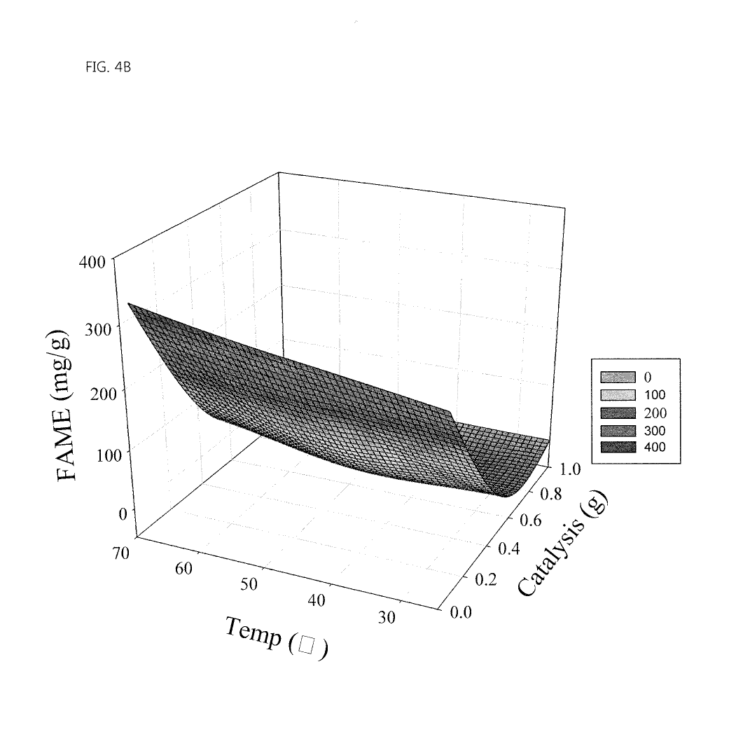

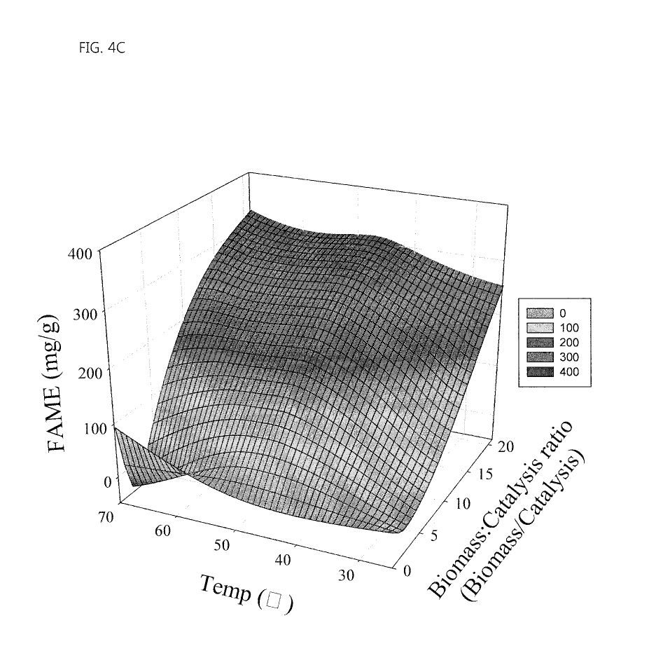

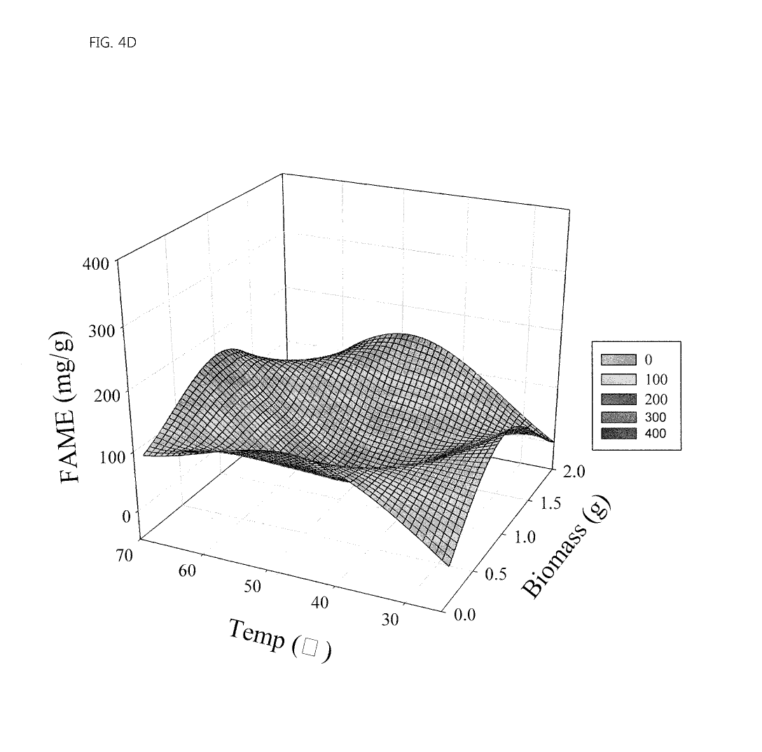

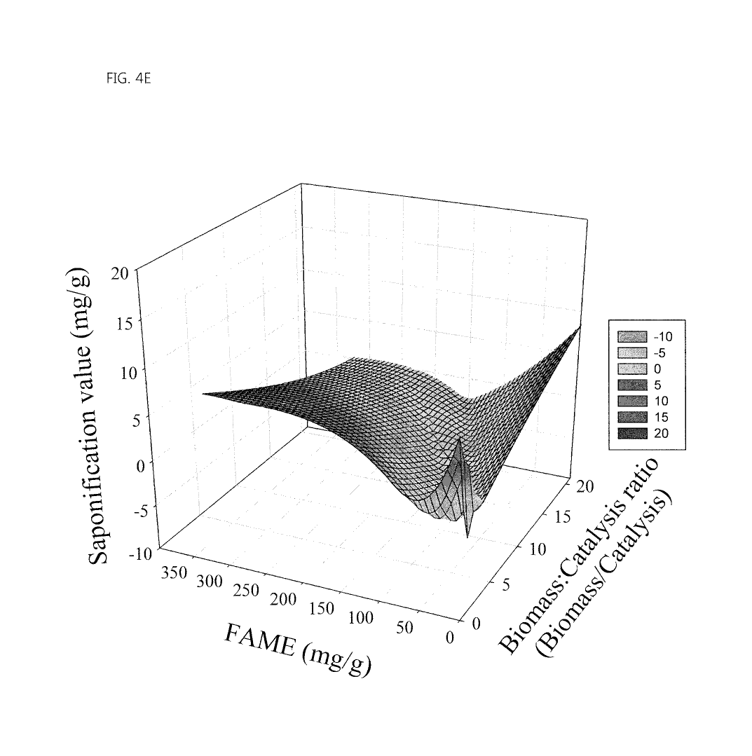

A pellet was added to a predetermined amount of methanol, dispersed homogeneously while stirring, and then RSM was performed using Minitab 14 in order to obtain in-situ transesterification efficiency according to an amount of a catalyst (G. Vicente et al.: Industrial Crops and Products 8 (1998) 29_{—}35) (FIG. 4).

As a result, the amount of the FAME could be estimated according to the amount of the catalyst, the amount of the biomass, and the reaction temperature. Also, it was found that there was no significant difference in the amount of the biodiesel produced in a temperature range of 4° C. to 70° C., and therefore the reaction may be performed efficiently at room temperature (i.e., 25° C.). Further, it was found that an increase in an amount of the biomass has no significant effect on the amount of the FAME produced (FIGS. 4B and 4D). However, the amount of the biodiesel showed a pattern with respect to the amount of the catalyst in which the yield of the FAME increased as the amount of the catalyst was reduced. In an attempted regression analysis of a model used in RSM in order to identify optimal catalyst conditions, it was found that the model was not suitable for identifying optimal conditions for transesterification. It was found that the optimal conditions for producing biodiesel lie outside of the range of conditions for carrying out RSM. It could be inferred from these two results that in-situ transesterification could be performed without a catalyst.

### Example 3

### Identification of Transesterification According to Amount of Catalyst Through RSM Analysis

In-situ transesterification efficiency according to the amount of catalyst was measured using biomass dispersed in methanol through RSM.

Specifically, 0.1 g of a pellet (wet biomass) obtained in Example <1-1> was added to 100 mL of methanol and dispersed while stirring for 1 hour. The biomass dispersed homogeneously in methanol was added to a 500 mL double jacket reactor, 0.00 g, 0.01 g, 0.02 g, 0.05 g, 0.10 g, 0.20 g, 0.50 g, 1.00 g, 2.00 g, and 3.00 g of NaOH were added thereto as a catalyst, respectively, and transesterification was performed while stirring at 300 rpm at 25° C. (room temperature). Further, types and amounts of FAMEs produced were identified after transesterification.

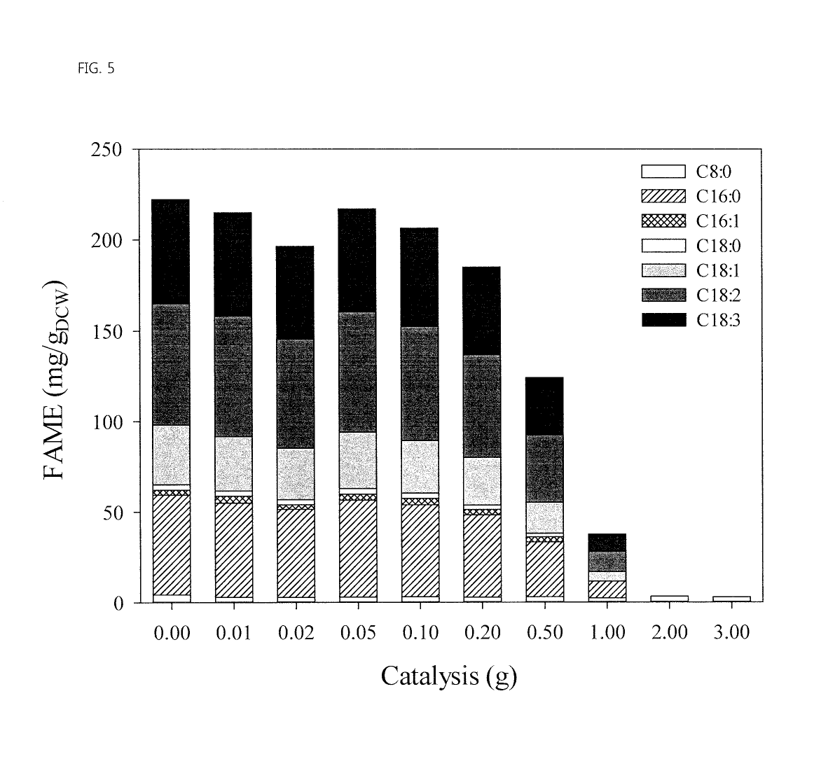

As a result, like the RSM analysis result, the amount of the catalyst and the amount of the FAME produced were inversely proportional to each other. When the amount of the catalyst was 0.20 g or less, similar amounts of the FAME were produced. Particularly, when the amount of the catalyst was 0 g (i.e., catalyst-free), it was found that the FAME was produced with high efficiency (FIG. 5)

For reference, in Example <1-2>, a wet biomass, methanol, and a catalyst were mixed and reacted, and dispersion of the pellet (wet biomass) in methanol was not performed. In Example <3>, a pellet was dispersed homogeneously in methanol, followed by performing reaction. Generally, it is known that diffusion of a solvent such as methanol (simultaneously reactant) in a biomass is a rate-limiting step that determines a reaction rate and efficiency in in-situ transesterification of a wet biomass. Therefore, when methanol is dispersed homogeneously in a pellet through the steps of the Examples, a FAME is produced rapidly and with high efficiency without a catalyst.

As described above, dried biomass particles aggregated together such that methanol was prohibited from permeating into cells, and thus extraction efficiency of lipid components in cells was reduced significantly. However, in the case of the present invention, since the amount of methanol is high relative to a wet biomass, methanol may sufficiently permeate into cells. Further, methanol is dispersed in the wet biomass, which helps with extraction of lipid components in cells.

### Example 4

### Identification of Biodiesel Produced in Optimal Reaction Condition Determined Through RSM Analysis

In order to identify applicability of various microorganisms in the process for producing biodiesel of the present invention, transesterification was performed using a yeast biomass in a reaction condition obtained through RSM analysis.

Specifically, the yeast Yarrowia lipolytica (provided by Biological Resource Center (BRC), Korea) was cultured for 14 days under light irradiation of 120 μmol m⁻² s⁻¹ in a YM medium in a 2 L bottle while air was fed at a rate of 0.1 v/v/m. A dry cell weight of the cultured yeast was measured. The cultured yeast culture medium was centrifuged at 4000 rpm for 5 minutes using a 50 mL conical tube, and then the supernatant was removed to obtain a pellet (with a moisture content of 82 to 85 wt %). A portion (with a dry weight of 0.5 g) of the pellet was subjected to transesterification at 300 rpm for 60 minutes at room temperature (25° C.). Subsequently, the amount of the biodiesel produced was identified by the method of Example <1-3>.

As a result, 0.5 g of a yeast biomass was converted into 224.82 mg/g of the amount of the FAME produced, which corresponds to a conversion efficiency of 22% or more per unit biomass (FIG. 7).

Therefore, it was found that the process of the present invention without drying and lipid extraction steps can be applied to a biomass of various microorganisms in addition to microalgae.

Through the method of producing biodiesel according to the present invention, biodiesel can be produced with a high yield through a simple process that does not include drying and lipid extraction steps, large amounts of biodiesel can be effectively produced without a catalyst under optimal reaction conditions, and thus production cost is considerably reduced.

The method of producing biodiesel of the present invention is simpler than an existing process, and can be used to produce biodiesel or a byproduct thereof, because biodiesel is effectively produced without a catalyst.

While the invention has been shown and described with reference to certain exemplary embodiments thereof, it will be understood by those skilled in the art that various changes in form and details may be made therein without departing from the scope of the invention as defined by the appended claims.

What is claimed is:

1. A method of producing biodiesel, comprising:
1) culturing microorganisms and centrifuging the culture broth to obtain a pellet;
2) adding the pellet of step 1) to an alkyl alcohol and performing a transesterification reaction; and
3) extracting a fatty acid methyl ester (FAME) from the reaction product of step 2).

2. The method of producing biodiesel of claim 1, wherein the microorganisms of step 1) are at least one selected from the group consisting of microalgae, yeast, bacteria, and fungi.

3. The method of producing biodiesel of claim 1, wherein the pellet of step 1) has a moisture content of 80 wt % to 98 wt %.

4. The method of producing biodiesel of claim 1, wherein the alkyl alcohol of step 2) is added in an amount of 10 to 10,000 mL per 1 g of dry weight of the pellet.

5. The method of producing biodiesel of claim 1, wherein the alkyl alcohol of step 2) is methanol or ethanol.

6. The method of producing biodiesel of claim 1, further comprising mixing the pellet with the alkyl alcohol, and dispersing the pellet in the alkyl alcohol after adding the pellet of step 2) to the alkyl alcohol.

7. The method of producing biodiesel of claim 1, further comprising adding a catalyst to the pellet during the transesterification reaction of step 2).

8. The method of producing biodiesel of claim 7, wherein the catalyst is a solid catalyst.

9. The method of producing biodiesel of claim 8, wherein the solid catalyst is an alkali catalyst, a metal oxide, or an alloy catalyst.

10. The method of producing biodiesel of claim 9, wherein the alkali catalyst is at least one selected from the group consisting of sodium hydroxide, potassium hydroxide, calcium hydroxide, magnesium hydroxide, aluminum hydroxide, barium hydroxide, iron hydroxide, lithium hydroxide, zinc hydroxide, nickel hydroxide, tin hydroxide, cobalt hydroxide, chromium hydroxide, ammonium hydroxide, zirconium hydroxide, titanium hydroxide, tantalum hydroxide, hafnium hydroxide, niobium hydroxide, and vanadium hydroxide.

11. The method of producing biodiesel of claim 9, wherein the metal oxide is at least one selected from the group consisting calcium oxide, magnesium oxide, strontium oxide, barium oxide, iron (II, III) oxide, aluminum oxide, copper oxide, sodium oxide, silicon dioxide, titanium oxide, tin oxide, zinc oxide, zirconium oxide, cerium oxide, lithium oxide, silver oxide, and antimony oxide.

12. The method of producing biodiesel of claim 7, wherein the catalyst is added in an amount of 0.01 to 10 g per 1 g of dry weight of the pellet.

13. The method of producing biodiesel of claim 1, wherein the transesterification reaction of step 2) is performed at 4 to 60° C. and 50 to 350 rpm.

14. The method of producing biodiesel of claim 1, further comprising recovering a magnetic metal oxide using an electromagnet, subjecting the magnetic metal oxide to heat treatment, and continually reusing the recycled metal catalyst after the transesterification reaction of step 2).

15. Use of biodiesel produced by the method of any one of claims 1 to 14.
